(19) United States
(12) Patent Application Publication
(10) Pub. No.: US 2022/0249159 A1
(43) Pub. Date: Aug. 11, 2022

(54) Systems and Devices for Ablation with Bendable Electrodes

(71) Applicant: SHANGHAI OPTIPULSE BIOTECH CO.,LTD, Shanghai (CN)

(72) Inventors: Longteng BAI, Beijing (CN); Jiahong TAN, Shanghai (CN)

(21) Appl. No.: 17/653,045

(22) Filed: Mar. 1, 2022

Related U.S. Application Data
(63) Continuation of application No. PCT/CN2021/091678, filed on Apr. 30, 2021.

(30) Foreign Application Priority Data
Jul. 6, 2020 (CN) 202010638621.8
Jul. 6, 2020 (CN) 202021292858.7
Aug. 21, 2020 (CN) 202010852167.6
Aug. 21, 2020 (CN) 202021767064.1
Sep. 25, 2020 (CN) 202022132634.6

(51) Int. Cl. A61B 18/14; A61B 5/367; A61B 5/287; A61B 5/00

(52) U.S. Cl. CPC A61B 18/1492; A61B 5/367; A61B 2018/00077; A61B 5/6858; A61B 2018/1475; A61B 5/287

(57) ABSTRACT

The present invention provides systems and devices for ablation with bendable electrodes, including the system console of ablation energy, pacing and ECG unit as well as ablation catheter. The ablation energy is radio frequency (RF) or voltage pulse. The ablation catheter is connected to the system console of the ablation energy through a converter, and the ablation energy is transmitted to the ablation tissue through electrodes on the ablation catheter, leading to degeneration of tissue cells. The ablation catheter includes an elastically retractable spline basket made up of a plurality of extendable splines, each spline has at least one electrode attached on the surface. The bendable electrodes have good adaptability, and can automatically fit to configuration of different ablation tissue, thus overcoming the nondeformable issue of long traditional ring-shaped electrode, thereby achieving a better ablation effect. The distal end of the spline basket is also coupled with an annular catheter entering the pulmonary vein (PV). Using different configuration of electrodes contacting tissue and variable combination of the electrodes in spline basket and annular catheter for various discharge ablation patterns, local, linear, annular or uniformly distributed large-area irreversible lesion can be formed, thereby achieving the purpose of treating arrhythmia diseases such as atrial flutter, supraventricular tachycardia, and atrial fibrillation (AF).

### CROSS-REFERENCE TO RELATED APPLICATION

This application is a continuation of International Patent Application Number PCT/CN2021/091678, filed on Apr. 30, 2021, which claims the benefit and priority of Chinese Patent Application Number CN202010638621.8, CN202021292858.7 filed on Jul. 6, 2020, CN202010852167.6, CN202021767064.1, filed on Aug. 21, 2020, CN202022132634.6 filed on Sep. 25, 2020. The entire disclosure of each of the foregoing applications is incorporated herein by reference in its entirety.

### FIELD OF THE INVENTION

The present disclosure belongs to the field of medical devices, and relates to an ablation system with bendable electrodes, in particular towards the treatment of arrhythmia using ablation catheter.

### BACKGROUND OF THE INVENTION

Since the first implementation of cardiac ablation in 1969, there have been numerous innovations and rapid developments in the field. Ablation was first used in treating supraventricular tachycardia with auxiliary pathways and pre-excitation syndrome. Today, ablation is applied in the treatment for atrial flutter, atrial fibrillation (AF), and ventricular arrhythmia.

The purpose of ablation is to destroy potential arrhythmia tissues, and form transmural and continuous permanent lesion. Percutaneous catheter ablation, which employs radio-frequency ablation (RFA) or cryoablation to achieve the pulmonary vein (PV) isolation in atrial tissue, has become a widely accepted interventional method for treating AF. Other energy forms developed for catheter ablation include microwave, high-intensity focused ultrasound, low-intensity collimated ultrasound, laser, low-temperature energy, and heated saline.

Radio-frequency (RF) energy is currently the most commonly used energy source. RF heats tissues by resistance and conducts the heat to deeper tissues to induce lesion.

Irreversible electroporation (IRE) is a rapidly developing treatment method for solid tumors, and it has been approved by FDA. IRE may be a promising method for cardiac ablation. In particular, compared with RF, IRE can induce ablation lesion without the consequence of heat conduction. In this regard, it can retain the surrounding tissue structure. This kind of voltage pulse is more commonly known as Pulsed Field Ablation (PFA) in this field. For RFA and PFA, there are urgent technical problems to be solved to improve ablation efficiency, strengthen ablation safety, as well as achieve rapid, safe and effective treatment of arrhythmia and other diseases.

### SUMMARY OF THE INVENTION

In order to achieve the above purpose, the present disclosure provides the following technical scheme.

The present invention provides systems and devices for ablation with bendable electrodes, including the system console of ablation energy, pacing and ECG unit, and ablation catheter.

The ablation energy is RF or voltage pulse. The ablation catheter is connected to the system console of the ablation energy through a converter, and the ablation energy is transmitted to the ablation tissue through electrodes on the ablation catheter, leading to degeneration of tissue cells.

The ablation catheter with bendable electrodes is characterized by comprising a proximal portion, a middle portion called main body and a distal portion which are sequentially connected. The ablation catheter is connected to the system console of the ablation energy, and the ablation energy is transmitted to the ablation tissue through electrodes on the ablation catheter;

The distal portion of the catheter includes an elastically retractable spline basket, including a plurality of extendable splines with bendable electrodes.

In a preferred embodiment, the proximal portion of the ablation catheter includes a control handle; the middle main body is an elongated tube body, and the tube body has a hollow lumen structure, including the outer tube, the conductive wire, the pull wire and the guide wire lumen.

Preferably, the whole or distal portion or middle portion of the spline is the conductive coil or the conductive coil set on the outside of the insulating tube. Each conductive coil corresponds to a bendable electrode, and the electrodes on adjacent splines are selected for positive and negative pairing to induce voltage pulse discharge ablation; It can also be connected with a RF instrument for monopolar ablation or bipolar RF ablation (RFA).

The conductive coil is made of round wire or flat wire. and the coil formed by single-wire or multi-wire arrangement which is preferably 2-5.

The bendable electrodes are replaced by conductive woven mesh, and each woven mesh corresponds to one electrode.

The distal end of the spline is fixed on a guide shaft with an inner lumen, and the guide shaft is directly connected to the knob or push rod of the control handle in the proximal portion of the catheter through a pull wire. A plurality of splines in the distal portion can be formed into a spline basket through the control handle, or the spline basket can be retracted into an extended position.

Preferably, the proximal end of the spline basket is connected to a fastener at the middle main body of the catheter, and the fastener is connected to a control handle at the proximal portion through a pull wire. The spline basket can be deflected by the control handle and adjusted to different positions.

Preferably, the spline basket includes 4-12 splines, preferably 6-8 splines.

Preferably, the distal portion also includes an annular catheter connected to the distal portion of the spline basket, and the annular catheter is provided with different electrodes for mapping or discharge ablation;

Preferably, the number of electrodes on the annular catheter and the number of splines can be identical, and the electrodes of both can be selected for pairing to perform discharge ablation.

Preferably, the guide wire in the guide wire lumen of the catheter enters vessel to assist the spline basket in the positioning and fitting at the vessel ostium.

Preferably, the structure of the annular catheter is preferably a ring formed by one circular ring, and a cylinder or a spiral cone formed by more than two circular rings.

In another embodiment, the distal portion is formed into one ring or a plurality of rings or extended spiral structure. The outer diameter of the spiral structure is 5-40 mm, and the pitch is 5-20 mm. The spiral structure is provided with electrodes with the number of 3-25.

Preferably, the middle main body is an elongated tube body, and the tube body has a hollow inner lumen structure, including the outer tube, the guide wire and the guide wire lumen.

Preferably, the outer diameter of the spiral structure is preferably 8-25 mm, and the number of electrodes on the spiral structure is preferably 4-10.

Preferably, the material of electrodes is selected from one or more of platinum, platinum alloy, gold, copper, stainless steel, nickel-titanium alloy, titanium alloy and MP35N.

Preferably, the ablation catheter is provided with a saline lumen, where saline can be irrigated.

Preferably, the guide wire drives and straighten the spiral structure of annular catheter. The guide wire 321 in the catheter 210 in the middle main body controls the angle of the distal portion, which then enters the selected pulmonary vein (PV). After reaching an appropriate position, withdraw the guide wire 321 to make the straightened distal portion be restored to its original spiral structure, thus achieving the close fitting with the inner wall of the vessel.

Preferably, the distal portion includes one annular structure with a larger proximal annular diameter and 1-2 annular structures with a smaller distal annular diameter, and the distance between them is 5-20 mm.

Preferably, the distal portion includes one or more annular catheters with equal annular diameters.

Preferably, the distal portion includes a spiral catheter, which has an elastic structure. The number of electrodes on the spiral catheter is 4-10.

Preferably, the outer diameter of the distal catheter is 1-4 mm, preferably 1.5-3 mm; The proximal structure of the distal portion can be close to the PV ostium, and the distal end structure of the distal portion is a catheter staying in the vessel.

The technical benefits obtained in this invention:

The splines in the spline basket are arranged in a partial coil structure, an integral coil structure, or a woven mesh structure, so that the electrode can be bent, and the bent electrode can be better fitted to the lumen or tissue surface, thereby achieving a better ablation effect. Meanwhile, the electrodes cover larger area, and a larger ablation region is achieved. Additionally, the bendable electrodes have good adaptability, and can automatically fit to configuration of different sizes of vascular lumens or other lumens, thus overcoming the issue of size matching in the traditional spline basket electrode.

The ablation catheter includes a spline basket, and the distal end of the spline basket is also coupled with an annular catheter entering the PV. Except that the electrodes on the spline basket are paired for discharge ablation at the PV ostium, the electrodes on the annular catheter can also be paired for bipolar discharge ablation. In this regard, it can increase the ablation range from traditional annular ablation at the PV ostium to annular ablation in the PV and cylindrical ablation between the two rings, thereby rapidly expanding the ablation region and achieving longer-term effective PV isolation.

3) By selecting and controlling the electrodes in spline basket and annular catheter for discharge ablation, local, linear, annular or uniformly distributed large-area irreversible ablation lesion can be performed, thereby achieving the purpose of treating arrhythmia diseases such as atrial flutter, supraventricular tachycardia, and AF.

4) The guide wire or annular catheter can enter the PV through the guide wire lumen of the ablation catheter, and the guide wire with bent end or circular catheter is positioned in the PV to ensure that the spline basket can be better fixed at the PV ostium, the bendable electrodes on it can be better contacted with tissues, and the ablation efficiency of the PV ostium can be improved, thereby achieving a complete PV isolation. Further, the annular catheter can also detect the signal attenuation of PV isolation in time.

### BRIEF DESCRIPTION OF THE DRAWINGS

The drawings wherein are used to provide a further understanding of the present invention and constitute a part of the present invention. The illustrative embodiments of the present invention and their descriptions are used to explain the present invention, and do not constitute an improper limitation of the present invention. In the drawings:

FIG. 1 is an illustration of the overall structure of the ablation system in accordance with the principles of the present invention;

FIG. 2 is an illustration of an exemplary use of the spline basket in accordance with the principles of the present invention;

FIG. 3 is another illustration of an exemplary use of the spline basket in accordance with the principles of the present invention;

FIG. 4 is another illustration of an exemplary use of the spline basket in accordance with the principles of the present invention;

FIG. 5 is another illustration of an exemplary use of the spline basket in accordance with the principles of the present invention;

FIG. 6 is still another illustration of an exemplary use of the spline basket in accordance with the principles of the present invention;

FIG. 7 is yet another illustration of an exemplary use of the spline basket in accordance with the principles of the present invention;

FIG. 8 is an illustration of an exemplary use of the conductive coil in accordance with the principles of the present invention;

FIG. 9 is another illustration of an exemplary use of the conductive coil in accordance with the principles of the present invention;

FIG. 10 is still another illustration of an exemplary use of the conductive coil in accordance with the principles of the present invention;

FIG. 11 is another illustration of an exemplary use of the spline basket in accordance with the principles of the present invention;

FIG. 12 is still another illustration of an exemplary use of the spline basket in accordance with the principles of the present invention;

FIG. 13 is yet another illustration of an exemplary use of the spline basket in accordance with the principles of the present invention;

FIG. 14 is an illustration of an exemplary use of the annular catheter in accordance with the principles of the present invention;

FIG. 15 is another illustration of an exemplary use of the annular catheter in accordance with the principles of the present invention;

FIG. 16 is still another illustration of an exemplary use of the annular catheter in accordance with the principles of the present invention;

FIG. 17 is an illustration of an exemplary use of the basket and distal catheter in accordance with the principles of the present invention;

FIG. 18 is an illustration of an exemplary use of the distal annular catheter after extension in accordance with the principles of the present invention;

FIG. 19 is an illustration of an exemplary use of the extended guide wire in the spline basket in accordance with the principles of the present invention;

FIG. 20 is an illustration of an exemplary use of the distal portion of ablation catheter in accordance with the principles of the present invention;

FIG. 21 is another illustration of an exemplary use of the distal portion of ablation catheter in accordance with the principles of the present invention;

FIG. 22 is still another illustration of an exemplary use of the distal portion of ablation catheter in accordance with the principles of the present invention;

FIG. 23 is yet another illustration of an exemplary use of the distal portion of ablation catheter in accordance with the principles of the present invention;

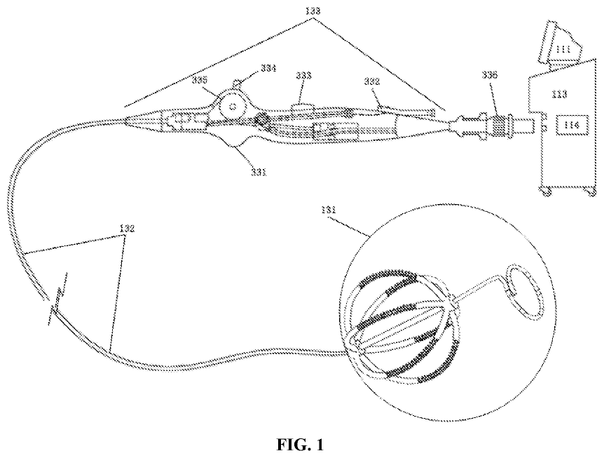

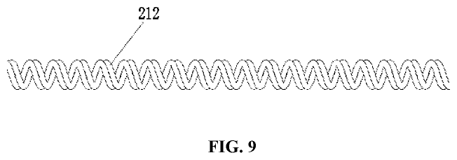

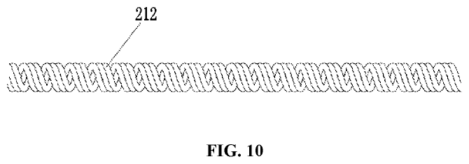

In the above FIGS. 1-19: 130. Ablation catheter; 131. Distal portion; 132. Middle main body; 321. Catheter; 322. Guide wire; 133. Proximal portion; 331. Control handle; 332. Connecting component; 333. Brake component; 334. Lever or knob; 335. Wire drawing component; 336. Connector; 210. Spline basket; 211. Spline; 212. Conductive coil; 213. The first insulating tube; 214. Fastener; 215. Guide shaft; 216. Inner insulated tubing; 220. Annular catheter; 221. The second insulating tube; 222. Electrode.

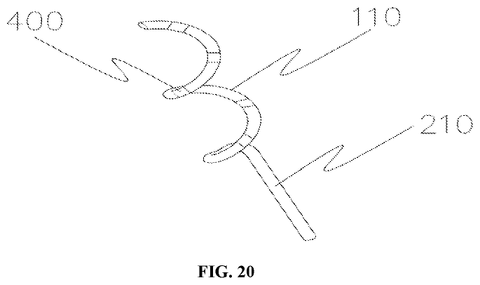

In FIGS. 20-23: 100. Distal portion; 110. Spiral catheter; 120. Annular catheter; 200. Middle main body; 210. Catheter in the middle main body; 300. Proximal portion; 310. Saline Luer connector; 320. Guide wire lumen; 321. Guide wire; 400. Electrode.

### DETAILED DESCRIPTION OF THE INVENTION

A more detailed understanding of the present invention will be more readily understood by reference to the following detailed description when considered in conjunction with the accompanying drawings wherein. It should be noted herein that the description of these embodiments is used to understand the present invention, but it does not constitute a limitation of the present invention.

The present invention provides an ablation system with bendable electrodes, including the system console of ablation energy, pacing and ECG unit, and ablation catheter.

The ablation energy source is RF or voltage pulse. The ablation catheter is connected to the system console of the ablation energy through a converter, and the ablation energy is transmitted to the ablation tissue through the electrodes on the ablation catheter, leading to lesion of tissue cells.

As shown in FIG. 1, the ablation catheter includes distal portion 131, middle main body 132, and proximal portion 133 which are sequentially connected.

The distal portion 131 of the catheter includes the elastically retractable spline basket, including a plurality of extendable splines with bendable electrodes.

The main body of the ablation catheter has a hollow inner lumen structure, including the outer tube, conductive wire, pull wire and guide wire lumen. The catheter with saline irrigation has saline lumen.

Further, the distal portion 131 includes a treatment head, such as spline basket 210 and/or annular catheter 220.

The middle main body 132 is the elongated tube, and the tube has a hollow inner lumen structure. The lumen is provided with the catheter, conductive wire, guide wire, etc.

The proximal portion 133 includes the control handle 331, which includes connecting component 332 for receiving guide wire or other therapeutic devices, and connector 336 connected with the handle body.

Further, the control handle 331 includes wire drawing component 335 for operating the treatment head of the distal portion 131, lever or knob 334, and brake component 333. The proximal end of the wire drawing component 335 may be anchored to component in communication with and responsive to lever or knob 334, such as cam. The brake component 333 is movably coupled to the proximal portion of the catheter and/or the control handle 331 to manipulate and move the treatment head component of the distal portion 131.

Further, the brake component 333 includes the sliding key, button, rotating rod or other mechanical structure movably connected to the control handle 331 or the ablation catheter 130.

In some embodiments, the control handle 331 has the slide bar, gear and pull wire structure, wherein the pull wire of a set of mechanisms is connected with the spline basket, and the spline basket is formed by rotating or pushing and pulling the control handle 331, or the spline basket is straightened and retracted to prepare for repositioning or ablating other PV The pull wire of another position control mechanism is connected to the proximal end of the spline basket, and the direction of the spline basket is controlled by the knob or push-button on the handle, making the spline basket be perfectly coupled to the PV ostium in different directions.

The catheter in the middle portion of the main body 132 is a woven mesh tube with excellent twist control, and the inner lumen of the mesh tube is the single-lumen or multi-lumen structure.

Further, the woven mesh tube comprises the inner lumen of insulating material, middle woven mesh and outer layer of insulating material. The inner lumen of insulating material is TPU or PEBAX, or polyimide, FEP, ETFE and PTFE with smaller friction coefficient and better insulation performance. The middle woven mesh is woven by stainless steel, Nitinol and other alloy wires. The outer layer is made of biocompatible electrical insulating materials such as TPU, PEBAX and nylon.

In another embodiment, if the woven mesh tube of the middle portion of the ablation catheter body 132 has a single-lumen structure, a guide wire lumen is formed by TPU, PEBAX, silicone rubber, polyimide, FEP, ETFE and PTFE tube. Additionally, the distal end extends into the spline basket 210. The proximal end enters the control handle 331 to form a guide wire lumen 332 with the hollow lumen on the luer connector, through which the guide wire 322 or the annular catheter 124 enters directly the PV.

In another embodiment, the distal portion 131 of the ablation catheter is a mesh-covered balloon, and electrodes embedded in the surface of the balloon complete discharge ablation.

In some embodiments, the distal portion of the ablation catheter 131 has an annular multipolar structure, and the catheter is configured to the PV ostium. It has an outer diameter of 1.5-5 cm, and the number of electrodes is 4-16, forming a complete PV isolation.

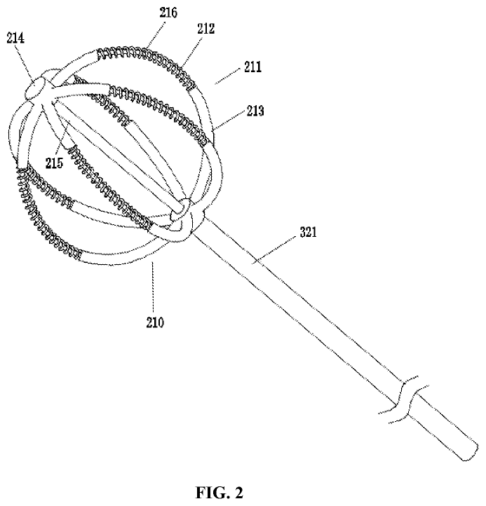

As shown in FIGS. 2-5, the treatment head of the distal portion 131 includes an extendable spline basket 210, which includes a plurality of flexible and extendable splines 211 with bendable electrodes, and the number of splines 211 is 2-12, preferably 4-8.

The main body of the spline 211 is an insulated side tube, and the conductive coil 212 is arranged at the distal end of the spline 211, and each conductive coil 212 corresponds to a bendable electrode. The conductive coil 212 preferably covers ⅓-½ of the length of the spline side tube, and the conductive coil 212 is closely coupled to the outer wall of the spline 211 side tube. The inner diameter of the conductive coil 212 is equal to or slightly larger than the diameter of the spline 211 side tube. The diameter of the side tube covered by the conductive coil is equal to or smaller than the diameter of the spline catheter body.

As shown in FIG. 2, there are 6 splines, and each conductive coil 212 covers the distal end of each spline, preferably covering ⅓-½ of the length of the spline side tube. The conductive coil 212 is closely coupled to the outer wall of the spline 211 catheter.

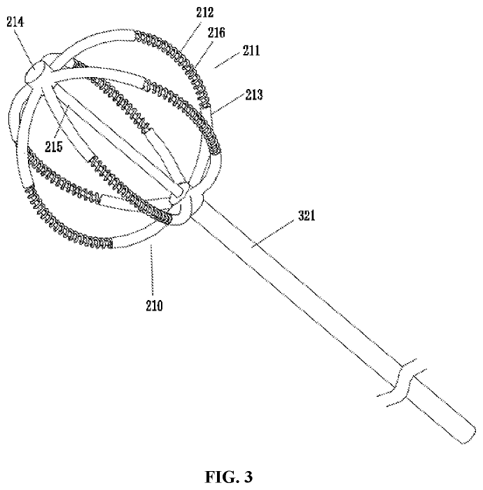

As shown in FIG. 3, there are 6 splines, and each conductive coil 212 covers the middle of each spline, preferably covering ⅓-½ of the length of the spline side tube. The conductive coil 212 is closely coupled to the outer wall of spline 211 side tube.

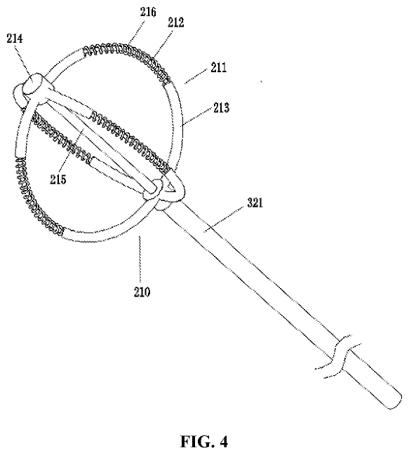

As shown in FIG. 4, there are 4 splines, and each conductive coil 212 covers the distal end of each spline, preferably covering ⅓-½ of the length of the spline side tube. The conductive coil 212 is closely coupled to the outer wall of the spline 211 side tube.

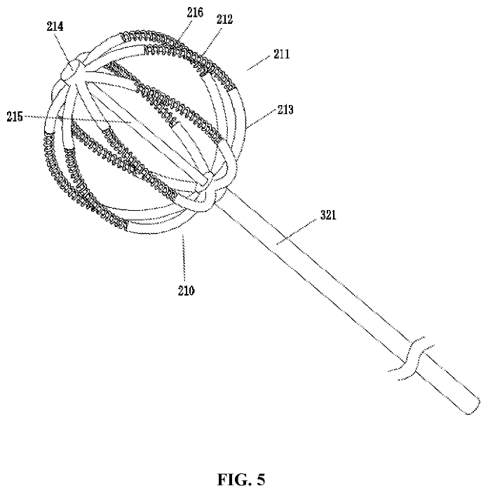

As shown in FIG. 5, there are 8 splines, and each conductive coil 212 covers the distal part of each spline, preferably covering ⅓-½ of the length of the spline side tube. The conductive coil 212 is closely coupled to the outer wall of the spline 211 side tube.

The system console of voltage pulse can address every electrode on the spline basket 210, and then select the electrode on the spline 211 for monopolar and bipolar discharge ablation.

The proximal end of the side tube of the spline 211 is fixed on the middle portion of the main body. The side tube is made of flexible polymeric insulating materials, including but not limited to polyimide, FEP, TPU, PEBAX, nylon and silica gel.

Further, the side tube 213 is internally provided with an insulated wire which is connected with the conductive coil 212, and the insulated wire is connected to the electrical socket of the control handle 331 through the catheter 321 of the middle portion of the main body 132.

Further, the proximal end of spline basket 210 is connected to catheter 321 of the middle portion of main body 321, and the distal end of the spline basket 210 is fixed on fastener 214 with inner lumen. A guide rod 215 is connected between the fastener 214 and the proximal end of the spline basket 210. The fastener 214 and the guide rod 215 are connected to the knob or push rod of the control handle 331 in the proximal portion by a pull wire. The spline basket 210 can be retracted or extended by the control handle.

Further, both ends of the conductive coil 212 in the spline 211 are respectively fixedly connected with the catheter 321 and the fastener 214 of the middle portion of main body 132 through the side tube 213.

Further, the conductive coil 212 is internally provided with an insulating side tube, and the diameter of the spline side pipe covered by the conductive coil is equal to or smaller than the diameter of the spline catheter.

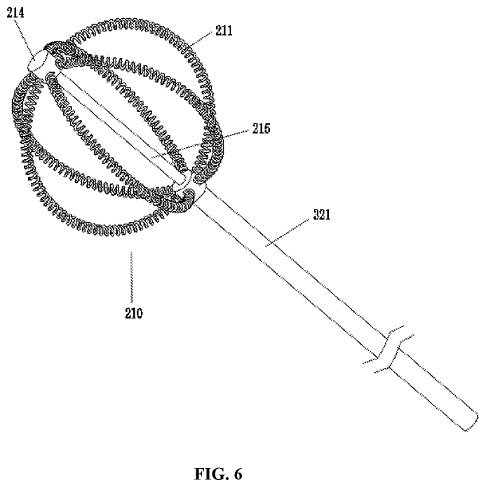

In another embodiment, as shown in FIG. 6, the whole spline 211 is conductive coil 212, and each conductive coil 212 corresponds to an electrode, which is connected with an energy source for ablation.

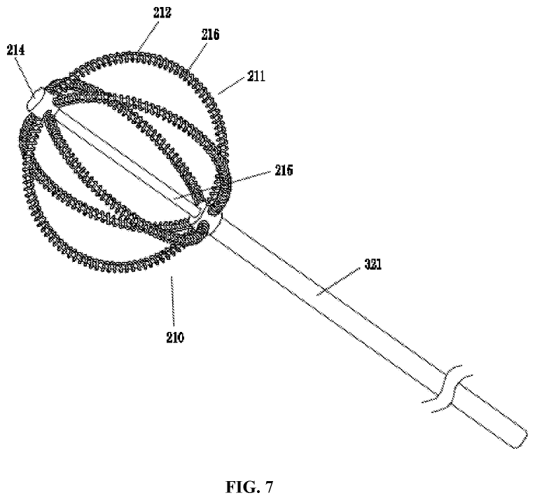

In another embodiment, as shown in FIG. 7, the spline 211 includes conductive coil 212 and inner insulating side tube 216. The conductive coil 212 is set on the inner insulating side tube 216, and each conductive coil 212 corresponds to an electrode and is connected with an energy source for ablation.

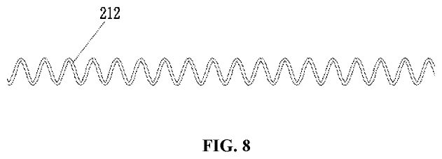

As shown in FIGS. 8, 9 and 10, the conductive coil 212 is a round wire or a flat wire. The coil formed by single-wire or multi-wire has wires that are uniformly and continuously arranged there-between, and there may also be gaps there-between. The multi-wire is preferably formed by connecting 2-5 coils in parallel, and most preferably three metal wires in parallel. The conductive coil 212 is a structural illustration of single wire, double wire and triple wire.

In some embodiments, the metal wires at both ends of the conductive coil are provided with insulating layers. The middle metal wire is the conductive region, and the length of the conductive area covers ⅓-½ of the length of the spline catheter.

In some embodiments, the conductive coil 212 is replaced by metal woven mesh, and each metal woven mesh corresponds to a bendable electrode, which is connected with an energy source for ablation.

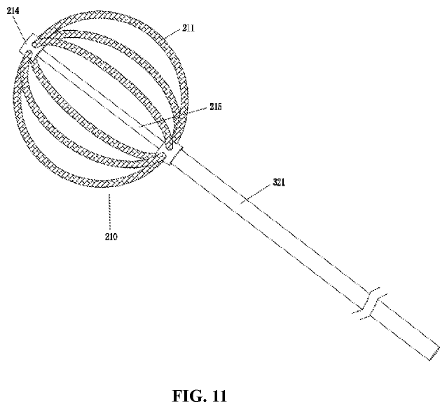

In some embodiment, as shown in FIG. 11, the conductive woven mesh is set on the insulating tube, and the conductive woven mesh is woven by metal wires with good flexibility and extensibility.

In some embodiments, the metal wires at both ends of the conductive metal woven mesh are covered with insulating layer. The middle metal woven mesh is formed as the conductive region, and the length of the conductive region covers ⅓-½ of the length of the spline side tube.

In some embodiments, the conductive woven mesh covers ⅓ to ½ of the length of the first insulating tube 213. Two ends of the metal woven mesh are connected with fixing pieces of annular electrode.

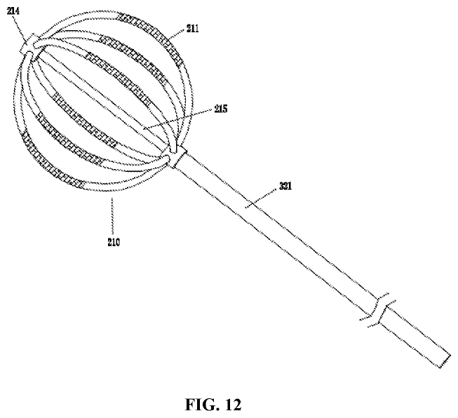

As shown in FIG. 12, In some embodiments, the conductive woven mesh covers the middle of each spline, preferably covering ⅓-½ of the length of the spline catheter. The conductive woven mesh is closely coupled to the outer wall of the spline 211 side tube.

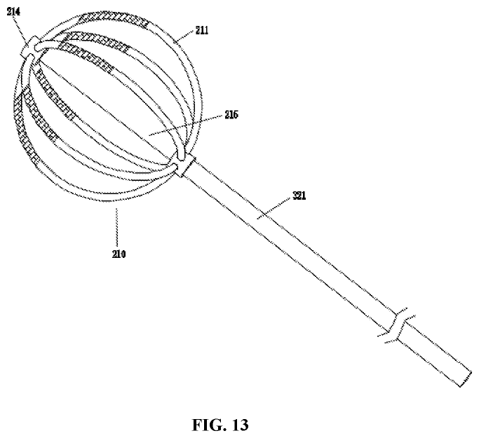

As shown in FIG. 13, In some embodiments, the conductive woven mesh covers the distal end of each spline, preferably covering ⅓-½ of the length of the spline catheter. The conductive woven mesh is closely coupled to the outer wall of spline 211 side tube.

The conductive coil 212 or conductive woven mesh is made of metal wire, including but not limited to metal platinum, platinum alloy (platinum iridium, platinum nickel, platinum indium, platinum tungsten), palladium and palladium alloy, gold, copper, stainless steel, nickel titanium alloy, titanium alloy and MP35N.

When there are a plurality of splines 211, in the state that the spline basket 210 is opened to form a basket shape, each spline 211 is uniformly distributed on a basket-shaped sphere of 360 degrees in three-dimensional space.

Further, the distal portion of the ablation catheter also includes annular catheter 220 connected to the distal end of spline basket 210. The annular catheter 220 includes insulating tube 221, and the outer wall of the annular catheter 220 has a plurality of electrodes 222.

The insulating tube 221 is made of flexible polymer insulating material, including but not limited to polyimide, FEP, TPU, PEBAX, nylon, and silicone. The insulating tube 221 is internally provided with insulated conductive wire, which is connected with electrodes 222 embedded on the surface of the annular catheter 220. The insulated wire passes through the fastener 214 and the guide shaft 215. Further, it is connected to the electrical socket of the control handle 331 through the catheter 321 in the middle main body 132.

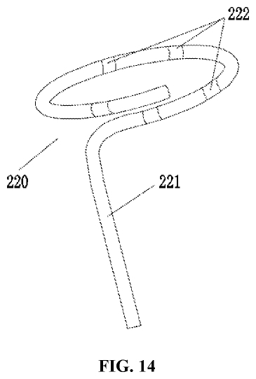

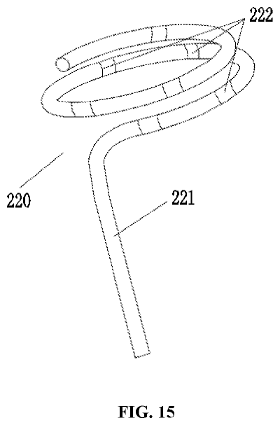

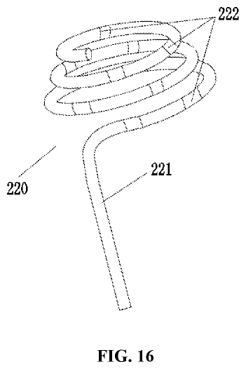

As shown in FIGS. 14, 15 and 16, the structure of the annular catheter 220 is preferably a loop formed by one circular ring (FIG. 14), and a cylinder or a spiral cone (FIG. 16) by more than two circular rings (FIG. 15)

In some embodiments, the annular outer diameter of the annular catheter 220 in the extended state is 10-30 mm, preferably 15-20 mm; The number of the electrode 222 is 5-15, preferably 6-10; The length of the electrode 222 is 1 to 4 mm, preferably 1.5 to 3 mm.

In another embodiment, electrodes 222 on the annular catheter 220 are bendable electrodes, and the bendable electrodes are distributed on or set outside the second insulating tube at intervals.

The annular catheter 220 can enter the PV, effectively detect the PV isolation, and can also discharge ablation. The annular catheter 220 enters the PV through the guide wire lumen of the ablation catheter.

In another embodiment, two adjacent electrodes 222 in the annular catheter 220 are set as anode and cathode, and complete voltage pulse discharge ablation in sequence or at simultaneously, thus forming complete PV isolation.

Further, the system console of voltage pulse 110 can address each electrode 222 of the annular catheter 220, and select electrodes 222 for positive and negative pairing to perform discharge ablation. Alternatively, it performs positive and negative pairs electrodes 222 of the conductive coil 212 on the spline basket 210 to conduct electric discharge ablation.

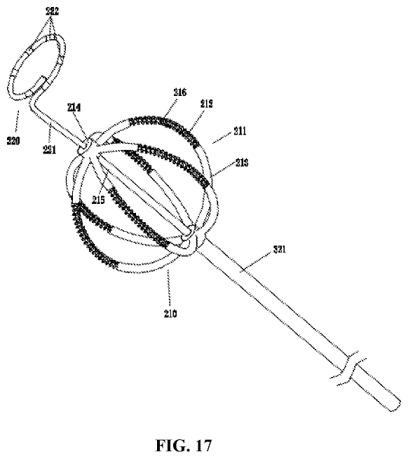

As shown in FIG. 17, the insulating tube 221 of the annular catheter 220 extends from the inner lumen of the guide shaft 215 of the spline basket 210 through the inner lumen of fastener 214. The proximal ends of a plurality of extendable flexible splines 211 are connected to the catheter 321 in the middle main body. The distal end of each spline 211 of the spline basket 210 is fixed on fastener 214 with inner lumen, and the guide shaft 215 can be extended and retracted from the catheter 321, thereby controlling the extension of the spline basket 210. The control handle of the proximal end can control the extension of the annular catheter 220 through the guide wire.

In another embodiment, the system console of voltage pulse 110 can address each electrode 222 of the annular catheter 220 and each electrode of the spline basket 210, and select adjacent electrodes to combine positive and negative electrode pairs for discharge ablation, thus performing three-dimensional cylindrical ablation.

Further, the number of electrodes 222 on the annular catheter 220 is the same as that of the spline 211, and the number of positive and negative electrodes is the same, thereby achieving the maximum discharge ablation effect.

Various combinations between different electrode arrays and addressable electrodes in the distal portion of the catheter contacting the tissue form various high-voltage pulsed electric field modes. By adjusting the position and potential of the electrodes, electrodes on the annular catheter 220 and electrodes on the spline basket 210 are discharged in multiple combinations to induce a larger range of discharge, and the ablation region of the discharge is more sufficient than that between two adjacent electrodes. Further, local, linear, annular, conical or uniformly distributed large-area irreversible lesions can be formed, thereby achieving the long-term efficiency of treatment of different arrhythmia diseases such as atrial flutter, supraventricular tachycardia and AF.

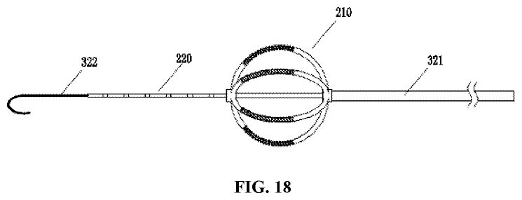

As shown in FIG. 18, the guide wire 322 in the catheter lumen extends out of the annular catheter 220, and the annular catheter 220 can be extended into a straight line, thus facilitating the movement in the blood vessel. After the guide wire 322 is withdrawn, the annular catheter 220 returns to a flexible annular, automatically configuring to the size of the lumen.

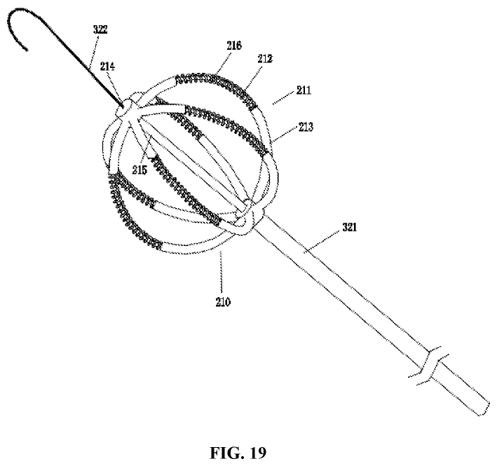

In some embodiments, as shown in FIG. 19, a guide wire 322 with bent end in the catheter lumen extends out of the spline basket 210 and enters the lumen to help the spline basket position and fit at the lumen opening. After the guide wire 322 is withdrawn, the flexible deformation of the annular ablation catheter is activated, automatically configuring to the size of PV.

In some embodiments, the distal portion 100 is formed into a ring or a plurality of rings or extended spiral structures. The outer diameter of the spiral structure is 5-40 mm, and the pitch is 5-20 mm. The spiral structure is equipped with electrodes 400 with the number of 3-25, and the length of the electrode 400 is 2-8 mm, preferably 3-5 mm.

Preferably, the outer diameter of the spiral structure is 8-25 mm, and the number of electrodes 400 on the spiral structure is 4-10.

The outer diameter of the tubing of the distal portion 100 is 1-4 mm, preferably 1.5-3 mm; The annular proximal end of the distal portion 100 can be close to the vascular opening. The annular distal end of the distal portion 100 is a catheter staying in the vessel, and a guide wire lumen 320 is also arranged in the catheter of the distal portion 100.

The guide wire 321 drives the spiral structure to expand and contract, thereby facilitating the straightening of the distal portion 100. The guide wire 321 in the catheter 210 in the middle main body controls the angle of the distal portion 100, which then enters the selected PV. After reaching an appropriate position, withdraw the guide wire 321 to make the straightened distal portion 100 be restored to its original spiral structure, thus achieving the close fitting with the inner wall of the vessel.

In some embodiments, as shown in FIG. 20, the distal portion 100 includes spiral catheter 110, which has the elastic structure. The number of electrodes 400 on the spiral catheter 110 is 4-10.

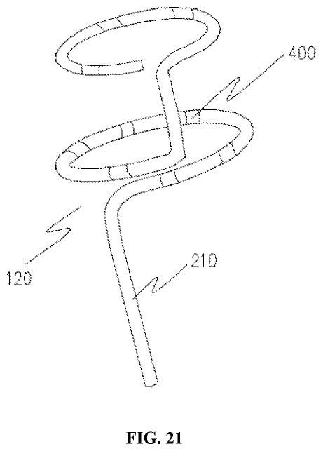

In some embodiments, as shown in FIG. 21, the distal portion 100 includes an annular structure 120 with a larger proximal annular diameter and an annular structure 120 with a smaller distal annular diameter, and the distance between them is 5-20 mm.

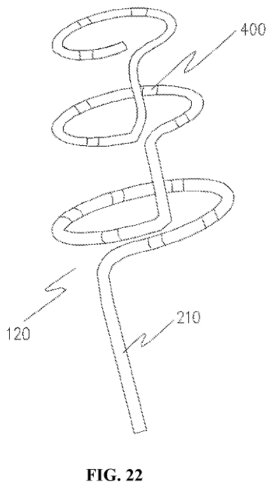

In some embodiments, as shown in FIG. 22, the distal portion 100 includes one annular structure 120 with a larger proximal annular diameter and two annular structures 120 with a smaller distal annular diameter, and the distance between them is 5-20 mm.

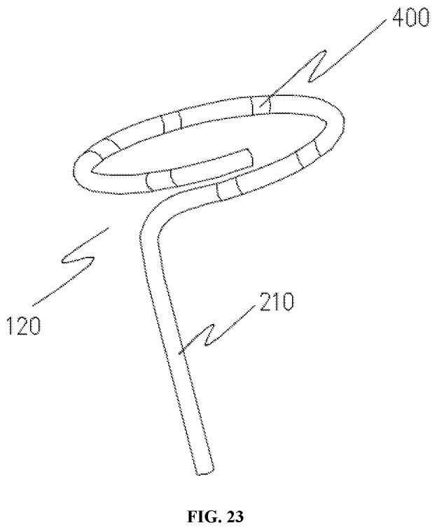

In some embodiments, as shown in FIG. 23, the distal portion 100 includes one or more annular structures 120 with equal annular diameters.

The above is only a preferred embodiment of the present invention, and is not intended to limit the present invention. The invention is subject to various modifications and variations for the technical personnel in the field. Any modification, equivalent substitution, improvement, etc. made within the spirits and principles of the present invention shall be included in the protection scope of the present invention.

We claim:

1. A medical system for ablation with bendable electrodes comprises the system console of ablation energy, pacing and ECG unit, and ablation catheter.
The ablation catheter with bendable electrodes is characterized by comprising a proximal portion, a middle portion called main body, and a distal portion which are sequentially connected. The ablation catheter is connected to the system console of the ablation energy; and the ablation energy is transmitted to the ablation tissue through electrodes on the ablation catheter;
the distal portion of the catheter includes an elastically retractable spline basket containing a plurality of extendable splines with bendable electrodes.

2. The medical system of claim 1, wherein the proximal portion of the ablation catheter includes a control handle; the middle portion is an elongated tube body, and the tube body has a hollow lumen structure, including the outer tube, the conductive wire, the pull wire and the guide wire lumen.

3. The medical system of claim 2, wherein the whole or distal portion or middle portion of the spline is the conductive coil or the conductive coil set on the outside of the insulating tube;
each conductive coil corresponds to a bendable electrode;
the electrodes on adjacent splines are selected for positive and negative pairing to induce voltage pulse discharge ablation; it can also be connected with a radio frequency (RF) instrument for monopolar ablation or bipolar RF ablation (RFA).

4. The medical system of claim 2, wherein the conductive coil is made of round wire or flat wire; and the coil formed by single-wire or multi-wire arrangement which has preferably 2-5 wires.

5. The medical system of claim 2, wherein the bendable electrodes are replaced by conductive woven mesh, and each woven mesh corresponds to one electrode.

6. The medical system of claim 2, wherein the distal end of the spline is fixed on a guide shaft with an inner lumen;
the guide shaft is directly connected to the knob or push rod of the control handle in the proximal portion of the catheter through a pull wire;
plurality of splines in the distal portion can be formed into a spline basket by the control handle, or the spline basket can be retracted into an extended position.

7. The medical system of claim 2, wherein the proximal end of the spline basket is connected to a fastener at the middle main body of the catheter, the fastener is connected to a control handle at the proximal portion through a pull wire, and the spline basket can be deflected by the control handle and adjusted to different positions.

8. The medical system of claim 2, wherein the spline basket includes 4-12 splines, preferably 6-8 splines.

9. The medical system of claim 2, wherein the distal portion also includes an annular catheter connected to the distal portion of the spline basket, and the annular catheter is provided with different electrodes for mapping or discharge ablation.

10. The medical system of claim 1, wherein the guide wire in the guide wire lumen of the catheter enters vessel to assist the spline basket in the positioning and fitting at the vessel ostium.

11. The medical system of claim 1, wherein the distal portion is formed into one ring or a plurality of rings or extended spiral structure;
the outer diameter of the spiral structure is 5-40 mm, and the pitch is 5-20 mm; the spiral structure is provided with electrodes with the number of 3-25.

12. The medical system of claim 1, wherein the distal portion includes one annular structure with a larger proximal annular diameter and 1-2 annular structures with a smaller distal annular diameter, and the distance between them is 5-20 mm.

13. The medical system of claim 1, wherein the distal portion includes one or more annular catheters with equal annular diameters.

14. The medical system of claim 1, wherein the distal portion includes a spiral catheter, which has an elastic structure. The number of electrodes on the spiral catheter is 4-10.
